(19) United States
(12) Patent Application Publication
(10) Pub. No.: US 2015/0212676 A1
(43) Pub. Date: Jul. 30, 2015

(54) Multi-Touch Gesture Sensing and Speech Activated Radiological Device and methods of use

(71) Applicant: AMIT KHARE, WAUKESHA, WI (US)

(72) Inventor: AMIT KHARE, WAUKESHA, WI (US)

(21) Appl. No.: 14/165,556

(22) Filed: Jan. 27, 2014

(51) Int. Cl. G06F 3/0484; A61B 6/00; G06F 3/16; G06F 19/00; G06F 3/041; G06F 3/0488

(52) U.S. Cl. CPC G06F 3/0484; G06F 3/041; G06F 3/04883; G06F 3/167; G06F 19/321; A61B 6/462; G06F 2203/04104

(57) ABSTRACT

Multi-touch gesture sensing and speech activated device for use with radiological workstations and method of use. This device may be capable of displaying medical DICOM compliant images. The device will be able to communicate with the attached workstation(s) via one or more controllers capable of receiving speech commands through a microphone and also capable of receiving multi-touch gestures from either hand via a touch screen containing sensing areas.

### FIELD OF INVENTION

The present invention relates to healthcare and more specifically, but not by way of limitation, to the field of radiology and radiological workstations.

### BACKGROUND OF THE INVENTION

Computer Information Technology is becoming increasingly ubiquitous within the radiology domain but the challenge is to provide radiologists with efficient and intuitive means for viewing and analyzing radiological images without affecting their quality of work. Although touch and speech are natural interfaces for human-computer interaction but conventional input devices like keyboards and mouse are primary devices for human-computer interaction even though these input devices are plagued with intrinsic limitations in simultaneous usage while performing multiple activities at the same time.

Radiologists use specialized software to view and analyze medical images stored in a Picture Archiving and Communication System (PACS) and then dictate out detailed observation reports based on the observations made while analyzing images. These observation reports are made available to referring physicians for further diagnosis but intrinsic limitations of conventional input devices limit a radiologist's ability to efficiently and effectively analyze medical images and simultaneously create diagnostic reports.

The present invention relates to multi-touch gesture sensing, speech activated devices and more specifically but not by way of constraining to multi-touch gesture sensing, speech activated devices for use with one or more components such as a radiological workstation connected to a PACS. The device empowers radiologists to access and analyze medical images as well as dictate observation reports efficiently and effectively. In some embodiments, the present invention may be targeted to a multi-touch gesture sensing, speech activated device connected to a radiological workstation for radiological image display and analysis. The device including (a) a touch pad that is communicatively connected to the radiological workstation via a device controller, the touch pad has one or more sensing areas for receiving touch gestures from either hand of the user, (b) wherein the touch gestures received via the touch pad execute functions controlling the medical imaging application executable on the radiological workstation, (c) a microphone that is communicatively connected to a radiological workstation via a device controller, (d) wherein the speech commands received via the microphone from the user execute functions controlling the medical imaging application executable on radiological workstation. A medical imaging application is used to search and display radiological images for analysis and creation of diagnostic reports.

According to additional embodiments, the present invention could be directed to radiological workstations with the ability to display radiological medical images. The radiological workstation may have (a) some memory for storing different software's like the medical imaging software, device driver software, the operating system etc., (b) a central processing unit for executing different software's like device driver software, operating system software etc., (c) a controller coupled to the workstation with a multi-touch gesture sensing device, (d) a multi touch gesture sensing device includes: (1) a touch pad connected to a radiological workstation with one or more sensing areas for receiving touch gestures from either hand of the user, and (2) wherein the touch gestures received via the touch pad perform functions controlling the imaging application, (e) a controller coupled with the workstation with a microphone, (f) the speech commands received via the microphone from the user perform functions controlling the imaging application. The medical Imaging application is used to display radiological images for analysis and creation of diagnostic reports.

According to the latest disclosure, methods for controlling the medical imaging application executing on a radiological workstation are described herein. Medical imaging application has functions for search and analysis of medical images as well as for creation of diagnostic reports. The methods may include the following steps: (a) receiving a request to display a singular radiological image or a group of radiological images for analysis. The request could be a touch gesture received via the multi touch sensing device or a speech command received via a microphone. The multi touch gesture sensing speech activated device including: (1) a touch pad connected to a radiological workstation with one or more sensing areas for receiving touch gestures from either hand of the user, and (2) wherein the touch gestures received via the touch pad execute functions controlling the medical imaging application. (3) The speech commands will be received via a microphone connected to the radiological workstation, and (4) wherein the speech command received via the microphone execute functions controlling the medical imaging application.

### BRIEF DESCRIPTION OF THE DRAWINGS

FIG. 1 is a block diagram of an exemplary architecture for practicing various embodiments of the invention.

FIG. 2 is the perspective view of a radiologist workspace displaying multiple high resolution monitors and input devices like keyboard and mouse.

FIG. 3 is the perspective view of a radiologist workspace displaying multiple high resolution monitors and a hand held microphone along with a touch pad for receiving gestures.

FIG. 4 is a block diagram of device driver software for connecting a multi touch gesture sensing speech activated device with a radiological workstation.

FIG. 5a is a block diagram for a touch pad which could be used for passing gestures to the medical viewing application via an Application Programming Interface.

FIG. 5b is a block diagram for a microphone showing controls which could be used for passing speech commands to the medical viewing application via an Application Programming Interface.

FIG. 6 is a flow chart describing a method for analyzing at least one image and dictating out an observation report.

### DETAILED DESCRIPTION OF THE INVENTION

Before any embodiments of the invention are explained in detail, it is to be understood that the invention is not limited in its application to the details of construction and the arrangement of components set forth in the following description or illustrated in the accompanying drawings. The invention is capable of other embodiments and of being practiced or of being carried out in various ways.

Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect. Also, electronic communications and notifications may be performed using any known means including direct connections, wireless connections, etc.

It should be noted that a plurality of hardware and software based devices, as well as a plurality of different structural components may be utilized to implement the invention. In particular, when terms such as controller, control unit, engine, module, etc. are used in the following detailed description, it should be understood that these terms can represent non-transitory computer-readable medium encoded with instructions that when executed by a processing unit result in various actions and computations. Furthermore, and as described in subsequent paragraphs, the specific configurations illustrated in the drawings are intended to exemplify embodiments of the invention and that other alternative configurations are possible.

Multi-touch gesture sensing speech activated device and methods of use are provided herein. In a typical radiology workflow, images are captured by different modalities types such as Computed Radiography (CR), Computed Tomography (CT), Ultrasound (US), Magnetic Resonance (MR), Nuclear Medicine (NM) etc. and are then forwarded to a PACS over the network via the Digital Imaging and Communication in Medicine (DICOM) protocol. PACS stores these images to its attached primary tier or secondary tier file storage system and make these archived images available to specialized image viewing software for radiologists to view and analyze these images and create reports either by dictation or typing. These reports will further be used by referring physicians in diagnosing aliments. The archived images can also be retrieved on demand from a PACS by a radiologist, a referring physician or other specialists.

The specialized image viewing software used by radiologist to view and analyze images is installed on a computer referred to as the “workstation”. The difference between a radiologist's workstation and a normal computer is that a workstation is a normal computer that comes installed with specialized medical image viewing software and has specialized high resolution monitors connected to it. These specialized high resolution monitors help radiologist's distinguish and analyze each and every detail in a medical image. Lack of visibility of any details within an image may severely hinder a radiologist's ability to record appropriate observations thus affecting diagnosis and overall patient care.

Keyboards and mouse are primary devices for human-computer interaction. A radiologist's workstation is communicatively connected to these conventional input devices. These input devices pass user input to the medical image viewing software as well. The software interprets the input and accordingly displays or manipulates medical images.

Reviewing and analyzing medical images is a highly skilled craft requiring careful analysis and attention to detail. It can also be a very tedious and time consuming task since medical images need to be carefully analyzed from multiple view points and can also be annotated with notes and comments.

With the advent of advanced medical imaging techniques, each study performed on a patient could have hundreds of medical images underneath it. Each of these images need to be evaluated either individually or collectively as found appropriate by a radiologist. For example, in case of MR images, these images may be analyzed three dimensionally requiring a radiologist to view them spatially with a high degree of concentration. Any lapse in concentration could result in an erroneous judgment on part of the radiologist which could result in a wrong diagnosis eventually affecting patient care. This slip-up in patient care could lead to a patient's death which may result in law suits against the concerned radiologist and the medical organization. Hence, a radiologist workstation and the medical imaging software should be designed in such a way that it complements a radiologist's skills and helps him concentrate for long periods of time without distractions.

Currently, A radiologist, for typing annotations on medical images or pressing a shortcut on the keyboard or starting/stopping a medical image playback, moves his eyes to locate the appropriate key(s) on the keyboard and then positions his hands on the keyboard to type annotations on medical images or press a shortcut on the keyboard or start/stop an image playback. The movement of eyes and hands diverts a radiologist's attention during image analysis potentially resulting in a wrong diagnosis.

Similarly, in order to use a mouse, the radiologist first has to locate the mouse, grab it, move the mouse pointer to the appropriate position on the screen and then click the primary/secondary mouse button to pass the input to the medical imaging software to perform a task.

The human eye is our window to the world around us. It behaves just like a camera and detects light reflected off of an object and focuses on it through an adjustable assembly of lenses to form an image. It then converts this image into a set of electrical signals, and transmits these signals to the brain. Although remarkable, one of the major limitations of the human eye is that the human eye cannot concentrate on two objects simultaneously. While viewing a near point object, the eye accommodates (focus) and then converges (turn inwards) and objects in the background blur out. Similarly, while viewing a far point object, the eyes accommodate (focus) and they diverge (turn outwards) and objects in the foreground blur out. In no circumstance, it is possible for the human eye to diverge and converge at the same time consequently the human eye cannot concentrate on two objects simultaneously.

Based on the dynamics of the human eye as described above, the movement of the mouse pointer creates a distraction for the human brain. The human brain starts processing objects which are in focus, the mouse movement in this case rather than concentrating on background objects which blur out, the medical image in this case. Due to the brain processing the mouse movement instead of medical images, the radiologist can inadvertently overlook analyzing vital information resulting in a wrong diagnosis.

Also, a radiologist's work is highly skilled and requires extensive use of conventional input devices for long periods of time therefore the risk of developing carpal tunnel syndrome is highly elevated.

The present invention attempts to solve the above stated problems by eliminating distractions caused by the use conventional input devices and also lowers the risk of developing carpal tunnel syndrome in radiologists by using natural interfaces for human-computer interaction like multi-touch gestures and speech can help radiologists effectively and efficiently analyze medical images without distractions thus improving patient care.

Multi-touch gestures are standardized motions used to interact with multi-touch sensing devices like touch pad etc. These multi-touch sensing devices recognize the presence of two or more points in contact with the surface. This plural-point awareness is often used in the implementation of advanced functionality such as activating predefined programs. The controller software receives these gestures and passes them to the medical image viewing software's application programming interface which then interprets these gestures and the appropriate action is executed on the medical image viewing user Interface.

Multi-touch gestures are a natural interface for human computer interaction and since gestures can be received by either hand via a touchpad. The touchpad can be conveniently placed next to either hand based on the radiologist's preference.

Medical image viewing software's are complicated software's with a complicated graphical user interface wherein functions are hidden under hierarchies of menu items. If gestures are created for each function associated with medical image viewing software, the number of gestures in the system will be very high and remembering the correct gesture associated with a particular function will be difficult and it will lead to confusion on usage. This confusion could cause the radiologist to regress to use of a conventional input devices for passing input to the medical image viewing software. This regressive behavior could lead to the original problem where a radiologist's concentration could get distracted due to the use of conventional input devices leading to a wrong diagnosis.

Speech is another natural interface for human-computer interaction where spoken words are given as an input commands to a computer program after processing. Currently, radiologists use speech recognition systems while dictating out observation reports via a microphone which is communicatively connected to a workstation. These observation reports are stored in the PACS, RIS or a HIS and then presented on demand to a referring physician.

In a typical healthcare IT workflow, a radiologist views one or more images while dictating reports and simultaneously marks one or more images as “key objects” which are significant images in the analyzed study of a patient. In order to perform activities like marking a key object while dictating an observation report, the radiologist's uses conventional input devices like a mouse or a keyboard to perform this activity. The use of standard input devices while performing simultaneous activities could result in a distraction for the radiologist and vital information could be overlooked resulting in a wrong diagnosis thus affecting patient care.

As is evident, independently either natural interface whether speech or multi-touch gesture alone does not provide much value to a radiologist in improving patient care but the present invention creates a sophisticated approach by using both natural interfaces collectively to rectify the inherent drawbacks in each natural interface and in turn creates value for the radiologist and improve patient care.

By using both natural interfaces collectively, there could be dedicated touch gestures for accessing certain commonly used functions for example a single tap for stopping a cine motion of images on a particular study etc. and other actions for example increasing the frame rate of image playback can be accessed through speech commands hence the issue of too many gestures in the system gets resolved. Touch gestures and speech commands can be customized by a radiologist as per his/her preference.

Also by using both natural interfaces collectively, a radiologist can now mark an image or a group of images as key object with a customized touch gesture for example simultaneous swipe of two fingers while dictating out an observation report via a microphone which is communicatively connected to a workstation. The use of customized touch gestures and speech commands will not result in a lapse in concentration due lack of eye and hand movements hence the chances of overlooking some vital information is highly diminished thus greatly improving patient care.

Therefore, a multi-touch gesture sensing and speech activated device may be provided to analyze medical images effectively and efficiently create radiological reports while avoiding the distractions caused by the use of standard input devices.

Due to a highly diminished dependence on conventional input devices, the incidence of carpal tunnel syndrome developing in radiologists is also greatly diminished.

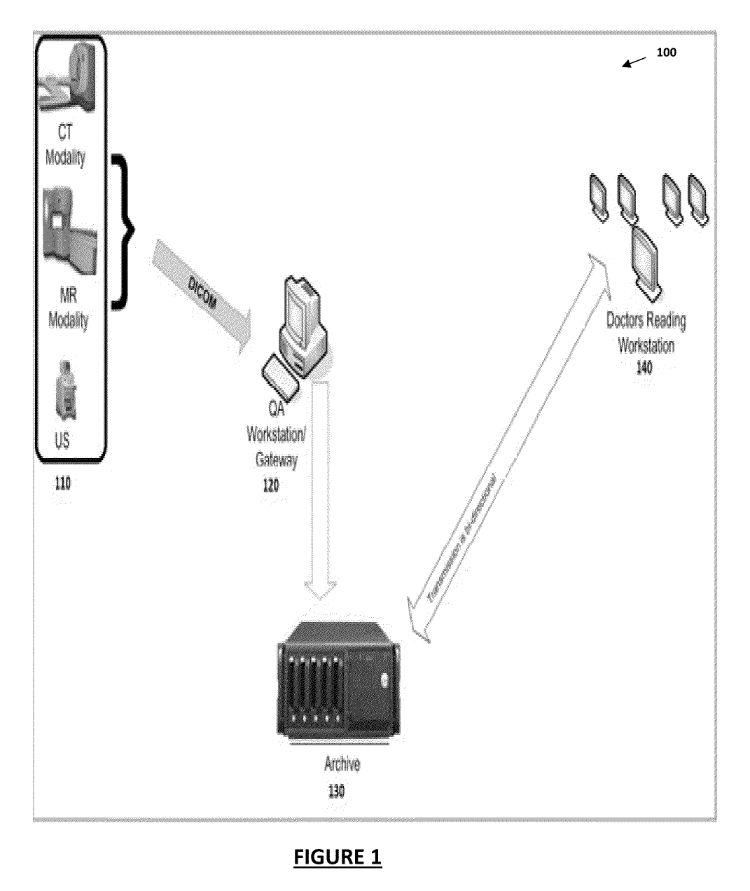

Referring now to the drawings, and particularly to FIG. 1, a block diagram of a typical architecture 100 for practicing various embodiments of the invention is shown resembling all or part of a picture archiving and communication system (PACS).

As shown in FIG. 1, different modalities 110 but not limited to a specific modality for example CT modality, MR modality or US modality etc. may be communicatively connected with a QC WorkStation 120. A QC WorkStation 120 is a workstation that provides a comprehensive set of specialized quality management tools along with a convenient user interface to guarantee the best possible image resolution for radiologists. The main user of a QC workstation 120 is the pre-assigned radiology technician who can modify the patient's image information, review images and perform specific image processing.

The QC WorkStation 120 may be communicatively connected with the Archive Server 130. The Archive Server 130 could be located in the same facility as the different modalities 110 and the QC WorkStation 120 or in a completely different facility and connected to each other over the network which may include but not limited to the internet or a LAN or a WAN etc.

A number of Reading Workstations 140 may be connected to the Archive Server 130 over the network which may include but not limited to the internet or a LAN or a WAN etc. facilitating a bi-directional transfer of radiological images, studies and/or reports.

It should be understood that one or more components of architecture 100 may function as per the specifications in the Digital Imaging and Communication in Medicine (DICOM) standard that governs the methods by which radiological images are obtained, stored and transmitted between devices.

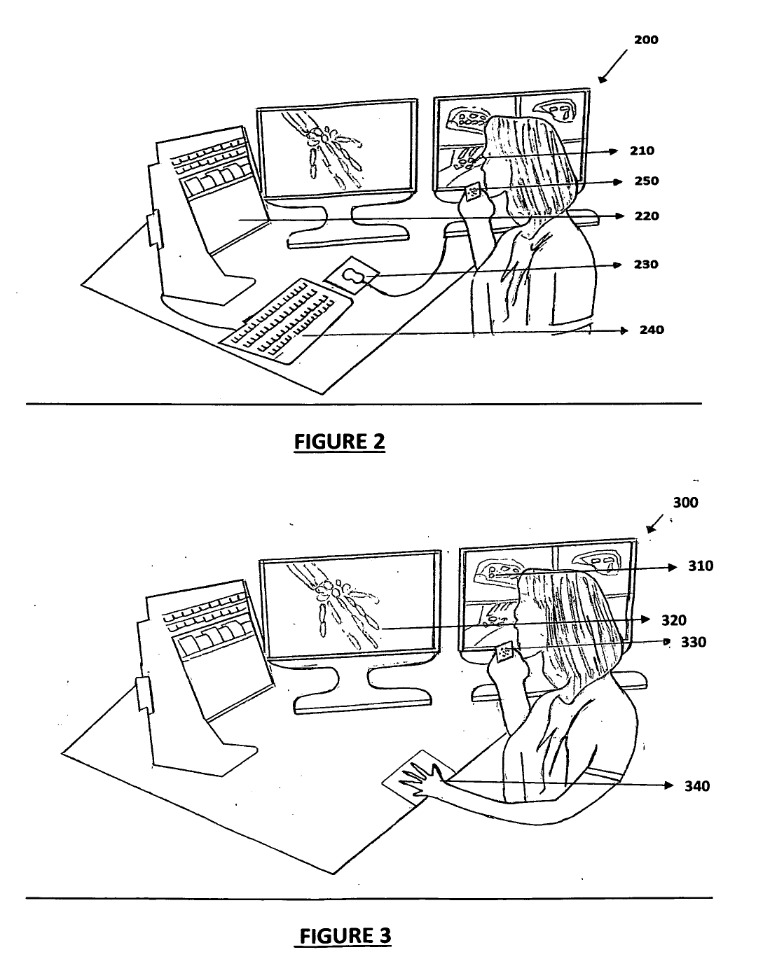

FIG. 2 shows a perspective view 200 of a radiologist's workspace displaying a multitude of high resolution monitors and input devices like keyboard and mouse. This pictorial representation of a conventional workstation 200 for a radiologist could serve as one of the workstations as part of architecture 100. There are multiple high resolution monitors 210 and 220 which could be used to display various images within a DICOM study conducted on a patient or could be used to display one or more interfaces generated by the user interface module, as discussed in greater details herein or both. The displayed study could be radiology or cardiology study but not limited to radiology or cardiology study. FIG. 2 also displays a keyboard 230 and a mouse 240 which are utilized to give input to the medical image viewing software. The software modules installed on the workstation interprets these inputs and accordingly display or manipulate medical images. The keyboard 230 is also be used by a radiologist or a cardiologist to type in a diagnostic report based on his observations made during image review whether reviewing them as static images or in a cine format where images are played at a pre-determined frame rate. Also shown in FIG. 2, is a microphone 250 which could be used by radiologist or cardiologist but not limited to a cardiologist or a radiologist to dictate observation reports based on the observations/findings discovered during image review whether reviewing images in a static fashion or in a cine format where images are played at a pre-determined frame rate.

FIG. 3 displays a perspective view of a radiologist's workspace displaying multiple high resolution monitors and a hand held microphone for dictating observation reports along with a touch pad for receiving gestures. There are multiple high resolution monitors 310 and 320 which could be used to display various images within a DICOM study conducted on a patient or could be used to display one or more interfaces generated by the user interface module, as discussed in greater details herein or both. The displayed images within the study could be radiology or cardiology study but not limited to radiology or cardiology study. FIG. 3 displays a microphone 330 which could be used to give speech command input to the medical image viewing software via a software controller 400. The software controller 400 installed on the workstation interprets these voice inputs and accordingly displays or manipulates medical images. It can also be used by a radiologist or a cardiologist to dictate a diagnostic report based on the observations made during image review. Also, displayed in FIG. 3 is a touch pad 340 which can be used to give gesture input to the medical image viewing software via the software controller 400. The software controller 400 installed on the workstation interprets these gesture inputs and accordingly displays or manipulates medical images.

The touch pad 340 could include a number of devices or assemblies capable of receiving input as gestures from either hand including but not limited to pinching, sliding, sweeping, tapping, single touch dragging etc. The touch pad may use any one of a number of commonly used technologies including but not limited to resistive, capacitive, strain gauge etc. Note that the human hand in FIG. 3 is not necessarily drawn to scale, for clarity of illustration.

The touch pad 340 may be communicatively connected to a workstation 140 via a number of commonly used network connections like Wi-Fi, Bluetooth, fire wire, Ethernet or any other wireless or wired connections.

A device driver application 410 may be installed on a device controller 400 or it might be installed along with a medical imaging application on the radiological workstation 140. A controller 400 may be utilized to communicatively connect the radiological workstation 140 with the touch pad 340 and the microphone 330.

The device driver application 410 may be adapted to translate touch gesture input received via the touch pad 340 into one or more functions within the medical imaging applications 440 associated with the workstation 140. The medical imaging application 440 may be adapted to allow a radiologist to analyze radiological images by executing a series of functions like view, annotate, open, close, save, scroll, pan, zoom, crop, flip, invert, adjust window level, sort, rotate, change layout, center, highlight, draw reference line, annotate, 3D render, select, mark key image, save key image, display all key images, or combinations thereof. This is not an exhaustive list of functions for the sake of brevity.

According to some embodiments, one or more modules may be included as part of the device driver application 410 and constituent modules may be executed by the central processing unit of the radiological workstation 140 which may be adapted to accomplish respective functionalities attributed thereto.

According to some embodiments, the device driver application 410 may include a user interface module 405, a gesture customization module 415, a gesture analysis module 420, a speech analysis module 425, a speech customization module 430, an application programming interface 435 passing appropriate commands to a medical imaging application 440.

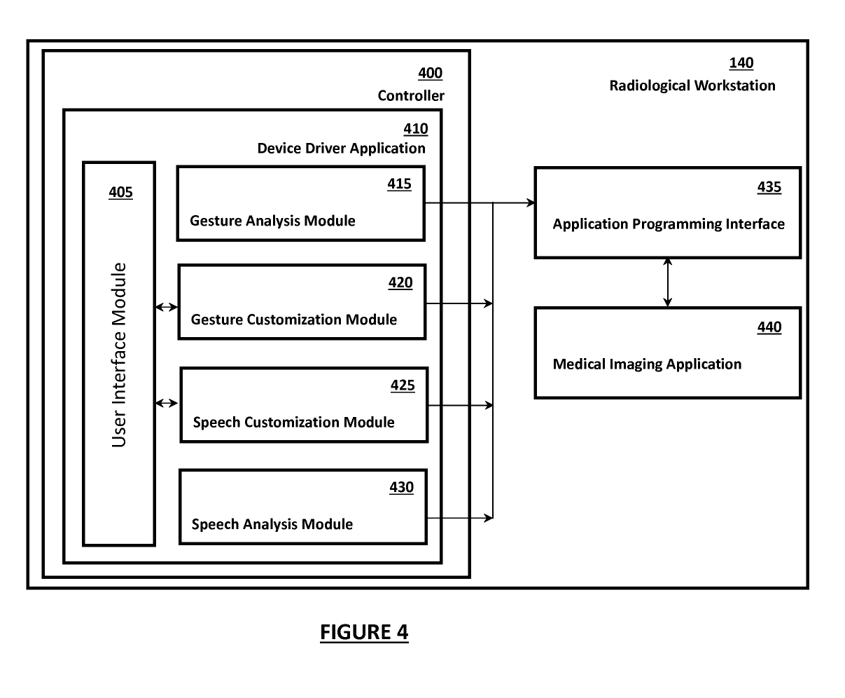

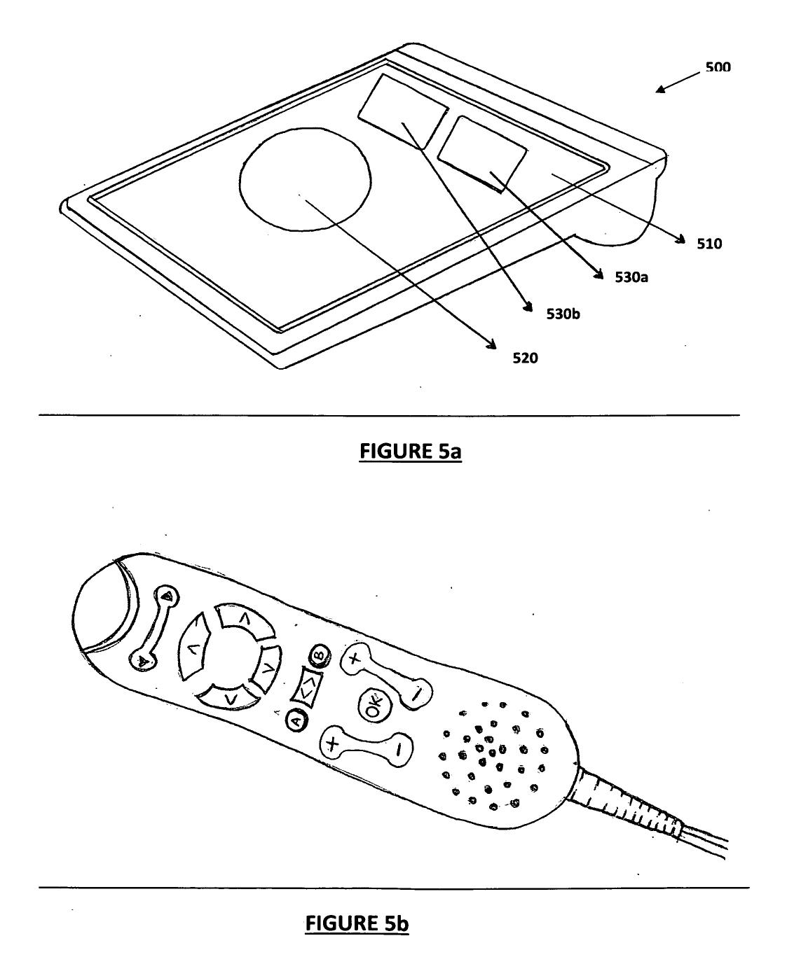

Referring now to FIGS. 4, 5A, and 5B collectively show components and internal connections of an exemplary system that is adapted to receive touch gestures and speech commands to control the execution of functions of the medical imaging application 440.

According to some embodiments, the touch sensing area 510 may include a circular sensing area 520 having a plurality of polygonal sensing areas 530a-b but not limited to polygonal sensing areas arranged in a pattern around the top portion of the circular sensing area 520 so as to allow for swiping and pinching touch gestures in addition to taping gestures, swiping gestures and other gestures. It should also be understood that plurality of polygonal sensing areas are limited to only two polygonal sensing areas in the touch sensing 510 but may include additional or fewer sensing areas within the touch sensing 510. The circular sensing area 520 is not limited to circular shape and could be in any polygonal shape.

Customized Touch gestures might be configured for to display links to objects such as a documents etc. or a help menu that would include a plurality of help related topics relative to functions of the medical imaging application or files residing in storage devices of the remote archiving server 130.

Touch gestures might also be configured using the Gesture Customization module 420 to access radiological studies/reports of a patient stored either locally on the radiological workstation 140 or remotely on remote archiving server 130, to label an area of interest on a particular radiological image within a radiological study, to allow the radiologist to perform generalized or specific searches both locally and remotely, for any one of a number of objects such as radiological images, documents etc.

A radiologist may utilize a number of functions available to him as part of the medical imaging application 440 to analyze radiological images and create radiological reports containing observations made during analysis of radiological images. These functions may include but are not limited to any of: open, close, save, scroll, pan, zoom, crop, flip, invert, level, sort, rotate, change layout, center, highlight, outline, draw reference line, annotate, 3D render, measure, erase, stack, brightness, contrast, reposition, select, key mark, key save, display all key images, etc., or combinations thereof. Gesture could be assigned to each of the functions listed above and their combinations thereof using the Gesture Customization Module 420. For example, in some embodiments, a simultaneous two-touch up-and-down gesture such as a single-fingered touch within the sensing areas 530a-b may result in scrolling through a radiological study for the currently selected patient in the radiologist work-list. Details of the work-list are not enumerated here but would be well known to one of ordinary skill in the art with the present disclosure before them.

Similarly, speech commands could be created and associated for each of the functions described above (for which gestures might have already been created) using the speech customization module 425. A radiologist might speak the voice commands into the microphone 330 associated with the radiologist workstation 140, which are then translated by the application programming interface 435 into an equivalent representation understood by the medical imaging application 440 and the appropriate function is performed. For example, in some embodiments, a speech command like “scroll” may result in scrolling down through the radiological images within a radiological study for currently selected patient on the radiologist work-list. Details of the work-list are not enumerated here but would be well known to one of ordinary skill in the art with the present disclosure before them.

The gesture customization module 420 and speech customization module 425 may be adapted to generate a list of available functions of the medical imaging application 440 and allow the radiologist to create and customize new gestures and voice commands with a particular function or group of functions.

Prior to utilizing the devices shown in FIGS. 5A and 5B, the device driver application 410 needs to be configured to pass voice commands and gestures to the medical imaging application 440 via the gesture analysis module 415 and speech analysis module 430.

The application programming interface 435 may be adapted to translate the gestures and speech commands defined by the radiologist, via the gesture analysis module 415 and speech analysis module 430, to pertinent functions of the medical imaging application 440. For the sake of brevity, as the device driver application 410 and the medical imaging application 440 are not limited to any particular coding language, a detailed discussion of the use of application programming interfaces will not be provided as the creation and use of application programming interfaces would be well known to one of ordinary skill in the art with the present disclosure before them.

Once a touch gesture from either hand is received via the touch pad 340, the touch gesture will be evaluated by the gesture analysis module 415. The gesture analysis module 415 may determine if the touch gestures received is associated with one or more functions of the medical imaging application 440. If the gesture analysis module 415 determines that one or more functions are associated with the touch gesture, the gesture analysis module 415 may communicate with the medical imaging application 440 via the application programming interface 435 to cause the medical imaging application 440 to execute the functionality attributed to the touch gesture or gestures received.

Similarly, once a speech command is received via the microphone 330, the speech command may be evaluated by the speech analysis module 430. The speech analysis module 430 may determine if the speech command received is associated with one or more functions of the medical imaging application 440. If the speech analysis module 430 determines that one or more functions are associated with the speech command, the speech analysis module 430 may communicate with the medical imaging application 440 via the application programming interface 435 to cause the medical imaging application 440 to execute the functionality attributed to the speech command received.

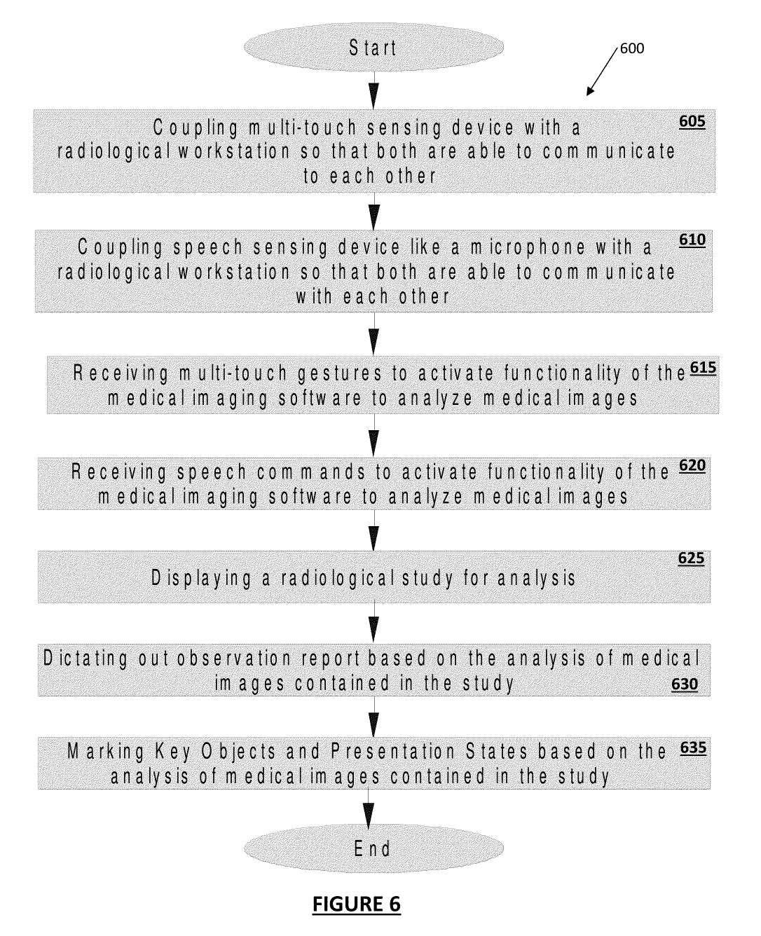

Referring now to FIG. 6, a method 600 for controlling a medical imaging application executable on a radiological workstation may include a step 605 of communicatively coupling a multi-touch sensing device with a radiological workstation. There may also be a step 610 of communicatively connecting a speech activated device with the radiological workstation. In some embodiments, the multi-touch sensing device and the speech activated device may be an integral part of the radiological workstation and hence steps 605 and 610 may not be necessary.

Next, the medical imaging application is executed on the radiological workstation and the multi-touch sensing device may receive a request to display a radiological study via touch gestures or speech commands received from the radiologist in step 615 and 620. The touch gestures may be received within any one of the sensing areas of the multi-touch sensing device. The speech command may be received through the microphone.

Once opened, appropriate touch gestures or speech commands may be received from the multi-touch sensing device in step 625 that are indicative of an analysis of radiological study by the radiologist.

In step 630, a radiological report may be created from the analyzed radiological images by receiving a digital signature corresponding to the radiologist. In step 635, the radiologist at the time of report dictation can use both the gestures and speech commands simultaneously to mark the key objects and other annotations on images while dictating out a report which is just not possible for a radiologist to do currently with use of conventional input devices without diverting his attention span and thereby enhancing the risk of over-looking areas within a radiological image or a group of radiological images necessary for appropriate diagnosis.

The signed radiological report may be stored locally on the radiological workstation or remotely on the remote archiving server. According to some implementations, the radiological reports may be directly communicated to a physician workstation located remotely from the radiological workstation or it may be sent to the hospital information system via the HL7 protocol for storage.

It is contemplated that any suitable features may be initiated and/or controlled via various gestures or speech. Some examples include but are not limited to invoking: a daily schedule and network, diagnosis request, image scan, viewing and analyzing case images, marking abnormal volumes, speech-to-text reporting, automated online searching for similar cases, opening an online reference case, calling the physician from a reference case for an audio and/or video conference, reviewing reports, etc.

While the present invention has been described in connection with a series of preferred embodiment, these descriptions are not intended to limit the scope of the invention to the particular forms set forth herein. It will be further understood that the methods of the invention are not necessarily limited to the discrete steps or the order of the steps described. To the contrary, the present descriptions are intended to cover such alternatives, modifications, and equivalents as may be included within the spirit and scope of the invention as defined by the appended claims and otherwise appreciated by one of ordinary skill in the art.

1. A Multi-Touch Gesture Sensing and Speech Activated device for use with a radiological work-station capable of displaying radiological images, the device comprising:
a touch screen communicatively connected with the radiological workstation via a controller wherein the sensing area adapted to receive touch gestures from a user; and
a microphone communicatively connected with the radiological workstation via a controller wherein it is adapted to receive speech commands from a user; and
wherein touch gestures received from the user via either hand via the multi-touch sensing device or speech commands received from the user via the microphone execute functions controlling a medical imaging application executable on the radiological workstation, the medical imaging application adapted to allow a user to analyze radiological images displayed by the radiological workstation and then dictate observation reports based their observations made during image analysis.

2. The device of claim 1, wherein the sensing area includes a circular area and a plurality of polygonal areas arranged in a pattern around an upper portion of the circular area.

3. The device of claim 1, wherein touch gestures execute functions controlling the medical imaging application via one or more application programming interfaces.

4. The device of claim 1, wherein speech commands execute functions controlling the medical imaging application via one or more application programming interfaces and can also be used.

5. The device of claim 3, wherein functions include any of open, close, save, scroll, pan, zoom, crop, flip, invert, level, sort, rotate, change layout, center, highlight, outline, draw reference line, annotate, 3D render, measure, erase, stack, brightness, contrast, reposition, select, key mark, key save, display all key images, and combinations thereof.

6. The device of claim 4, wherein functions include any of open, close, save, scroll, pan, zoom, crop, flip, invert, level, sort, rotate, change layout, center, highlight, outline, draw reference line, annotate, 3D render, measure, erase, stack, brightness, contrast, reposition, select, key mark, key save, display all key images, and combinations thereof.

10. The device of claim 1, wherein the radiological workstation is communicatively connected via a network control protocol with at least one of the radiological workstation, an image capturing device, a remote archiving server, and a physician workstation.

11. A radiological workstation capable of displaying radiological images, the workstation comprising:
a memory for storing a device driver application and a medical imaging application;
a processor for executing the device driver application and the medical imaging application; and
a controller communicatively coupled with the radiological workstation and a multi-touch sensing device that includes:
a touch screen adapted to receive touch gestures from a first hand of a user; and
a microphone or microphone communicatively coupled with the radiological workstation via a controller wherein it is adapted to receive speech commands from a user; and
wherein touch gestures and speech commands received from the user are translated into functions controlling the medical imaging application via one or more application programming interfaces, the medical imaging application adapted to allow a user to analyze radiological images displayed by the radiological workstation and create a radiological report indicative of the radio-logical images.

12. A method for controlling a medical imaging application executable on a radiological workstation, the medical imaging application having a plurality of functions that allow a user to analyze radiological images, the method comprising:
executing the medical imaging application;
receiving a request to display at least one radiological image, the request including touch gestures received from a multi-touch sensing device and speech commands from a microphone or a microphone. The multi-touch sensing device including:
a touch screen communicatively coupled with the radiological workstation via a controller, the touch screen adapted to display a work area that includes a sensing area adapted to receive touch gestures from the user; and
wherein touch gestures which include any of: pinch, swipe, slide, tap, and combinations thereof received from the user via the multi-touch sensing device and speech commands which include any of: launch study, show image, pan, zoom, and combinations thereof received from the user via microphone or microphone to execute functions controlling the medical imaging application;
and displaying at least one radiological image via a radiological workstation in response to a received touch gesture or speech command.

13. The method of claim 12, wherein prior to displaying at least one radiological image, the method includes displaying a radiological study that includes at least one radiological image and receiving touch gestures or speech command indicative of a selection of one or more radiological images from the radiological study.

14. The method of claim 12, further comprising updating the at least one radiological image based upon gestures received via the multi-touch sensing device or a speech command in response to displaying the at least one radiological image.

15. The method of claim 12, further comprising receiving audio notation corresponding to the at least one radiological image and associating the audio notation with the at least one radiological image in response to receiving one or more touch gestures via the multi-touch sensing device or speech commands via a microphone.

16. The method of claim 12, wherein functions controlling the medical imaging application include any of: open, close, save, scroll, pan, zoom, crop, flip, invert, level, sort, rotate, change layout, center, highlight, outline, draw reference line, annotate, 3D render, measure, erase, stack, brightness, contrast, reposition, select, key mark, key save, display all key images, and combinations thereof.

17. The method of claim 12, further comprising creating a radiological report by dictating observations and then storing analyzed radiological images as a record adapted to reside in a database communicatively connected with the radiological workstation.

18. The method of claim 19, wherein creating a radiological report further includes:
executing a dictation application in response to receiving a touch gesture via the multi-touch sensing device or speech command via the microphone;
receiving a dictated message corresponding to at least one radiological image via the dictation application; and
associating the dictated message with the at least one radiological image.

19. The method according to claim 19, wherein creating a radiological report further includes receiving input indicative of an electronic signature corresponding to a particular user.

20. The method of claim 21, further comprising establishing a peer-to-peer telecommunications link between the radiological workstation and a computing system via a touch gesture received from the multi-touch sensing device or a speech command received via a microphone.
